(19) United States
(12) Patent Application Publication
(10) Pub. No.: US 2008/0051771 A1
(43) Pub. Date: Feb. 28, 2008

(54) Ophthalmologic protective film

(75) Inventor: Hartmut E. Kanngiesser, Solothurn (CH)

Correspondence Address: OLIFF & BERRIDGE, PLC, P.O. BOX 320850, ALEXANDRIA, VA, 22320-4850, US

(73) Assignee: SIE AG SURGICAL INSTRUMENT ENGINEERING, Port (CH)

(21) Appl. No.: 11/892,369

(22) Filed: Aug. 22, 2007

Related U.S. Application Data
(60) Provisional application No. 60/840,693, filed on Aug. 29, 2006.

(30) Foreign Application Priority Data
Aug. 24, 2006 (EP) 06 405 367.1

(51) Int. Cl. A61F 9/009; A61N 5/067

(52) U.S. Cl. 606/4; 607/89

(57) ABSTRACT

A protective film (1) for protecting a cornea against direct contact with a laser applicator has a treatment area (2) of a first film thickness and a profile region (3) that is raised with reference to the treatment area (2) and whose film thickness is configured to be greater than the first film thickness. In the state in which the protective film (1) is arranged between the cornea and applied laser applicator, the treatment area (2) positions a first part of the cornea, which part is to be treated, in a focal region of the laser applicator, and the profile region (3) positions a second part of the cornea, which part is not to be treated, outside the focal region. The raised profile region (3) presses the eye tissue region not to be treated out of the cutting plane of the laser applicator such that the focused laser pulses cannot reach this tissue region, and therefore cannot inadvertently impair and damage it.

### TECHNICAL FIELD

The present invention relates to a protective film for protecting a cornea against direct contact with a laser applicator.

### PRIOR ART

Laser technology has already been in use for many years in opthalmologic surgery. In particular, it is possible by means of ultra short laser pulses, for example laser pulses of picosecond lasers with pulse widths of 1 ps to 10 ps (1 ps=10⁻¹² s) or laser pulses of femtosecond lasers with pulse widths of 1 fs to 1000 fs (1 fs=10⁻¹⁵ s), to cut what is known as a tissue flap on the outer surface of the cornea, something which was previously undertaken by microkeratomes with the aid of mechanical blades. In order to cut the eye tissue, it is necessary, on the one hand, to sharply focus the laser beam onto a few micrometers and, on the other hand, to place individual laser pulses accurately next to one another. In order to achieve a sufficiently high accuracy, the laser applicator and the zone of the eye that is to be treated must be coupled in a precise and stable fashion. However, direct eye contact with the laser applicator carries the risk of infectious material, for example viruses, being transferred from one patient to another if the laser applicator is not sufficiently disinfected, or of the eye tissue being mechanically damaged by the laser applicator.

Patent Application US 2005/0143718 describes a surgical method for treating an eye by means of ultra short laser pulses. In order to protect the eye against direct contact with a reference body of the laser applicator, a transparent film is pushed in between the eye and the reference body. Pulsed laser beams are projected by the laser applicator through the protective film and into the eye tissue. The protective film described in US 2005/0143718 cannot, however, prevent eye tissue outside the zone actually to be treated from being unintentionally destroyed by the laser beam in the event of improper operation, for example in the event of incorrect programming of the tissue zone to be treated.

### SUMMARY OF THE INVENTION

It is an object of the present invention to propose an improved protective film for protecting the cornea, in particular a human cornea, against direct contact with a laser applicator. It is to be clearly established in this case at this juncture that the term laser applicator does not mean simply a light projection system for pulsed laser beams, but also means a contact body that can be removed or is firmly connected to a light projector and is, for example, provided as an applanation body, reference body and/or exit window of the light projector, and through which the laser beams projected by the light projector are, in their final analysis, projected onto or into the eye tissue.

In accordance with the present invention, these goals are achieved, in particular, by the elements of the independent claims. Further advantageous embodiments emerge, moreover, from the dependent claims and the description.

The above-named goals are reached by the present invention more particularly in that the opthalmologic protective film for protecting a cornea against direct contact with a laser applicator has a treatment area of a first film thickness, for example a value in the range of 100 μm to 150 μm, and a profile region, raised with reference to the treatment area, which is configured to be thicker than the first film thickness (d₁). In the state in which the protective film is arranged between the cornea and applied laser applicator, the treatment area positions a first part of the cornea, which part is to be treated, in a focal region of the laser applicator, and the profile region positions a second part of the cornea, which part is not to be treated, outside the focal region. That is to say the (thicker) profile region raised with reference to the treatment area of the protective film presses the eye tissue region not to be treated out of the focal surface (focal plane, cutting plane) of the laser applicator such that the focused laser pulses cannot reach this region, and therefore cannot inadvertently impair and damage it. The protective film forms a sterile separation layer between the laser applicator and the eye. The protective film additionally fulfills the function of a spacing film, that is to say it serves as a spacer between the laser applicator and the human eye. As spacing film, the protective film determines the thickness of a tissue flap separated at least partially from the cornea, since the depth positioning of the focal surface of the laser applicator in the eye tissue is also determined by the thickness of the protective film.

In a preferred design variant, the treatment area is limited circumferentially by the profile region. The treatment area is, for example, arranged centered on the protective film, and the profile region is arranged concentrically with the treatment area. The treatment area is, for example, of circular configuration and has a diameter in the range of 8 mm to 11 mm. A treatment area delimited circularly by the profile region enables the cutting of tissue flaps in the cornea without the possibility of surrounding eye tissue being impaired by focused laser pulses.

In different design variants, the profile region is configured as a profile rim that is stepped away from the treatment area and has a second film thickness, or the profile region is configured such that it adjoins the treatment area with a gradually increasing film thickness, that is to say with a gradually increasing elevation, with reference to the treatment area. The profile rim has, for example, a second film thickness with a value in the range of 350 μm to 450 μm.

The profile region preferably has pressure equalizing openings that lead from the treatment area into an inner region of the profile region. In a preferred design variant, the pressure equalizing openings are embodied as grooves in the profile region, which lead, for example, from the profile wall into an inner region of the profile rim. When the laser applicator is applied to the eye, fluids (for example air and liquid) are forced between the protective film and the cornea. Moreover, gases are produced during cutting of the eye tissue by the laser pulses. The pressure equalizing openings can prevent overpressure between the protective film and the cornea, in particular between the profile region and the cornea. The pressure equalizing openings also make it possible to conduct away fluids that are forced between the protective film and the cornea when the laser applicator is applied, and/or are produced during cutting.

The side of the protective film that bears against the laser applicator in the applied state preferably has a flat surface. Particularly when the protective film is moistened, the flat surface permits movement on the laser applicator with little friction.

In one design variant, the protective film has a clamping rim surrounding the profile region and which is provided for fastening the protective film on a film carrier.

### BRIEF DESCRIPTION OF THE DRAWINGS

One design of the present invention is described below with the aid of an example. The example of the design is illustrated by the following enclosed figures:

FIG. 1 shows a schematic cross section of a protective film in the applied state between the cornea and a laser applicator.

FIG. 2a shows a plan view of the protective film, which illustrates pressure equalizing openings that lead from the treatment area into the inner region of the profile region.

FIG. 2b shows a cross section of the protective film along the cross sectional line C in FIG. 2a.

FIG. 3a shows a schematic cross section of the protective film with a profile region configured as a profile rim that is stepped away.

FIG. 3b shows a schematic cross section of the protective film with a profile region that adjoins the treatment area with a gradually increasing film thickness.

FIG. 3c shows a schematic cross section of the protective film with a further profile region that adjoins the treatment area with a gradually increasing film thickness.

### WAYS OF IMPLEMENTING THE INVENTION

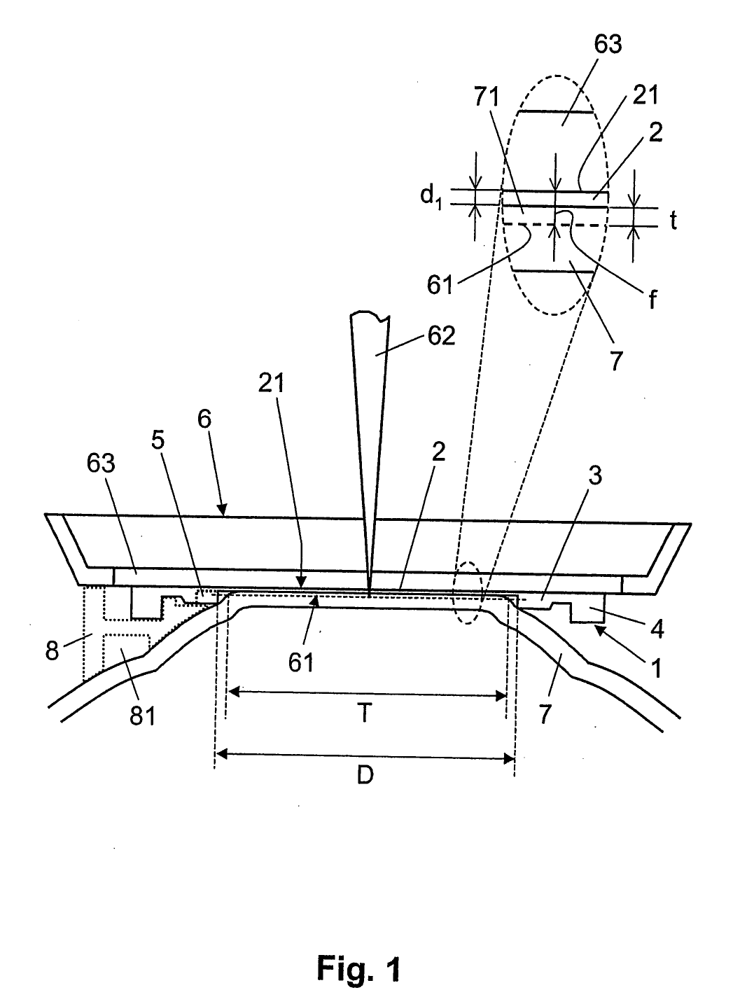

In FIG. 1, the reference symbol 6 denotes a laser applicator that is set up to project pulsed laser beams 62 onto a focal region 61, for example a flat focal surface (cutting plane). The laser applicator 6 comprises a permanently or removeably connected transparent contact body 63 that is, for example, provided as exit window, reference body and/or applanation body. The contact body 63 has, for example, a flat, concave or convex contact surface.

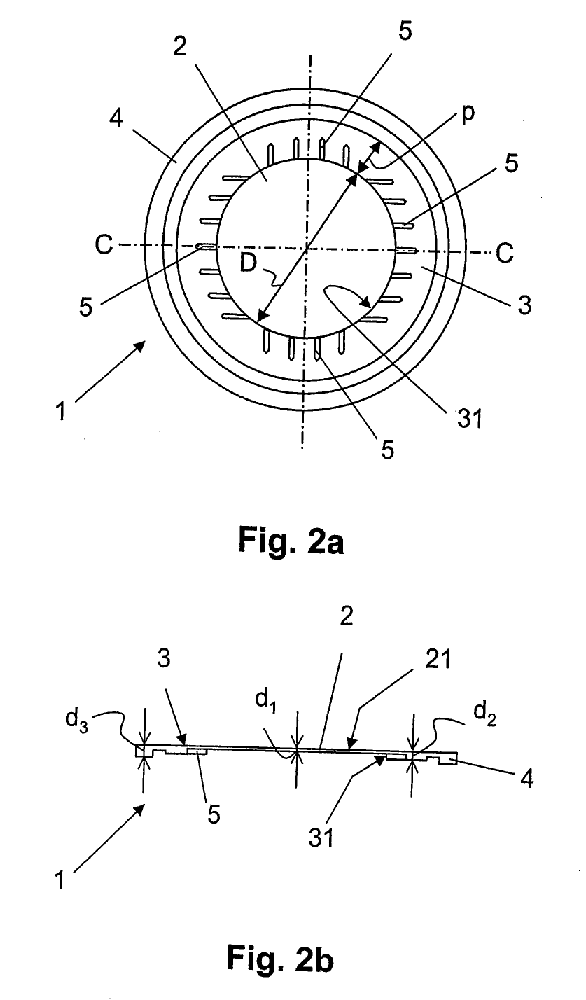

In FIGS. 1, 2a and 2b, the reference symbol 1 denotes a protective film that is preferably produced in one piece, using the injection molding method, from a flexible, biocompatible material that is highly transparent at least for the wavelength region around 1000 nm. The material is a thermoplastic elastomer, for example.

As may be seen from FIG. 1, the protective film 1 is inserted in the applied state between the laser applicator 6 and the eye, in particular the cornea 7. The protective film 1 has a side with a flat surface 21 that bears against the laser applicator 6 or against its contact body 63.

As illustrated in FIGS. 1, 2a, 2b, 3a, 3b and 3c, the protective film 1 comprises a treatment area 2, a rim profile that delimits the treatment area 2 and is denoted as profile region 3, and a clamping rim 4.

The treatment area 2 is preferably arranged as a film (film region) centered on the protective film 1, is of circular configuration and has a diameter D with a value in the range of 8 mm to 11 mm, and a thickness d₁ with a value in the range of 100 μm to 150 μm. In order to cut tissue flaps 71 of the cornea 7 of different flap thickness t and different flap diameter T, it is preferred to provide various protective films 1 with thicknesses d₁ of for example 110 μm±5 μm or 140 μm±5 μm, and with diameters D of, for example, 8.5 mm, 9.0 mm, 9.5 mm or 10.0 mm. Given a distance f of the focal region 61 (focal plane) from the laser applicator 6, or from the contact body 63 thereof, of 250 μm, for example, which distance determines the cutting depth, a tissue flap 71 with a flap thickness t of 140 μm (110 μm) results for a treatment area 2 with a thickness d₁ of 110 μm (140 μm).

The profile region 3 is preferably arranged concentrically with the treatment area 2, is of annular configuration and has, for example, an outer diameter of 14 mm to 16 mm.

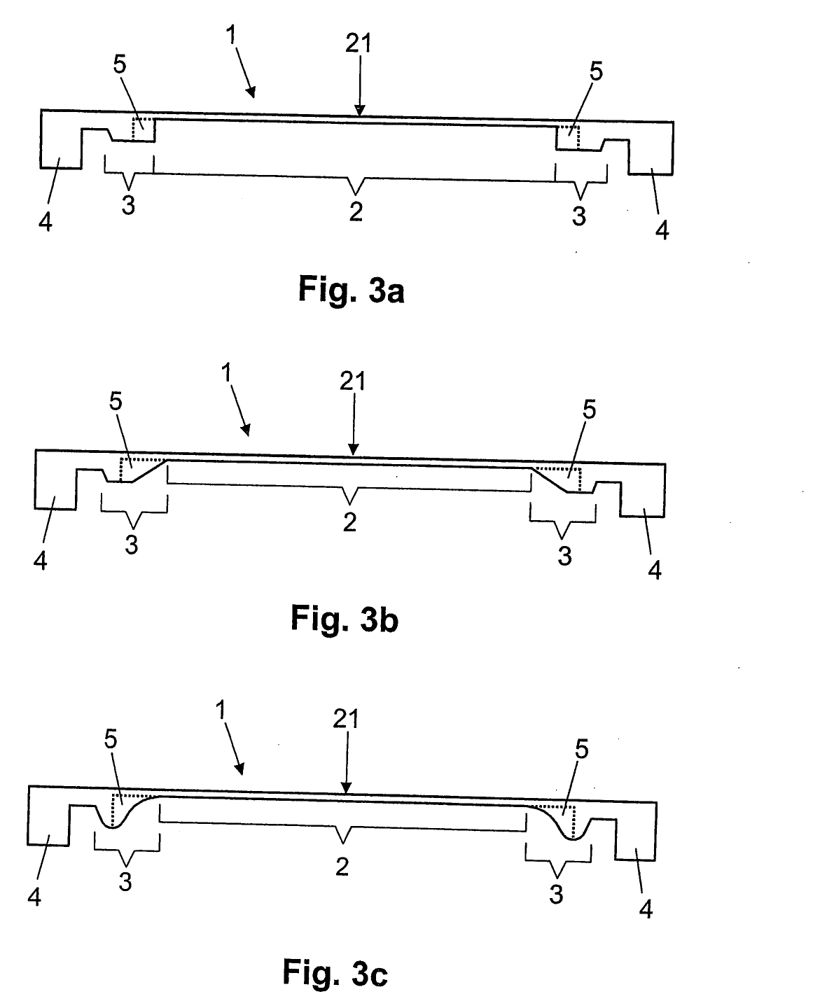

The profile region 3 is raised with reference to the treatment area 2. In the design variant in accordance with FIGS. 1, 2b and 3a, the profile region 3 is configured as a profile rim that is stepped away from the treatment area 2 and has, for example, a thickness d₂ with a value in the range of 350 μm to 450 μm. As is illustrated in FIGS. 3b and 3c, the profile region 3 is configured in further design variants such that its height profile continuously adjoins the treatment area 2, and its thickness increases gradually in the joining region with reference to the film thickness d₁ of the treatment area 2. That is to say, the profile region 3 adjoins the treatment area 2 with a film thickness gradually increasing with reference to the treatment area 2. It is therefore possible to configure the profile region 3 such that it adjoins the treatment area 2 with a stepped-away elevation (FIG. 3a), with a rectilinear and linearly increasing elevation (FIG. 3b) or with a curved, nonlinearly increasing (concavely, convexly) elevation (FIG. 3c).

The profile region 3 has pressure equalizing openings 5 that lead from the treatment area 2 into the inner region of the profile region 3, where they open into a circumferential groove for example. The pressure equalizing openings 5 are, for example, configured as grooves in the profile region 3 which, for example, lead in the profile rim from the profile wall 31 into the inner region of the profile rim 3, have a depth e=d₃−d₁ up to the level of the treatment area 2, and lead as far as approximately into the middle of the width p of the rim profile 3. In an alternative design variant, the pressure equalizing openings are recessed as a circumferential groove into the profile region 3.

As may be seen from FIG. 1, in the region of the profile region 3 the cornea 7 is pressed away out of the focal region 61 by the rim profile of the laser applicator 6 or by its contact body 63 such that the cornea 7 lies in this region outside the cutting plane of the laser and a tissue flap with a flap diameter of T<D results given a diameter D of the treatment area 2. The flap diameter T is determined by the shape and dimensioning of the profile region 3. Since the profile region 3 is thicker around the treatment area 2 than the distance f of the focal plane 61 from the surface of the laser applicator 6 or the contact body 63 thereof, the laser beam 62 therefore cuts out of the cornea 7 into the profile region 3 of the disposable protective film 1.

The clamping rim 4 seals the protective film 1, is preferably arranged concentrically with the profile region 3 and treatment area 2, is of annular configuration and has, for example, an outer diameter of 17 mm to 20 mm and a thickness d₃ with a value in the range of 450 μm to 550 μm. As illustrated in FIG. 1, the protective film 1 is fastened on a film carrier by means of the clamping rim 4. The protective film 1 is clamped, in particular, by means of the clamping rim 4 via an annular shoulder of a schematically illustrated suction ring 8, and thereby fitted removeably on the suction ring 8. The pressure equalizing openings 5 are configured and arranged such that they open into corresponding openings in the suction ring 8. In one design variant, the pressure equalizing openings 5 are connected to the suction ring 8 such that they communicate fluidically with the vacuum chamber 81 of the suction ring 8.

The protective film 1 can also be designed without a clamping rim 4 and be permanently connected to a disposable suction ring.

Only a circular concentric configuration and arrangement of the treatment area 2 and the profile region 3 have been described here, but a person skilled in the art will understand that other arrangements and shapings are also possible. For example, the treatment area 2 may be of oval or rectangular configuration, and/or be delimited rectilinearly by the profile region 3 on one or more sides.

1. A protective film for protecting a cornea against direct contact with a laser applicator, defined by a treatment area of a first film thickness that, arranged between the cornea and applied laser applicator, positions a first part of the cornea in a focal region of the laser applicator, and by a profile region that is raised with reference to the treatment area and, arranged between the cornea and applied laser applicator, positions a second part of the cornea outside the focal region.

2. The protective film as claimed in claim 1, wherein the treatment area is limited circumferentially by the profile region.

3. The protective film as claimed in claim 1, wherein the profile region is configured as a profile rim that is stepped away from the treatment area and has a second film thickness.

4. The protective film as claimed in claim 1, wherein the profile region adjoins the treatment area with a film thickness increasing gradually with reference to the treatment area.

5. The protective film as claimed in claim 1, wherein the profile region has pressure equalizing openings that lead from the treatment area into an inner region of the profile region.

6. The protective film as claimed in claim 5, wherein the pressure equalizing openings are embodied as grooves in the profile region.

7. The protective film as claimed in claim 1, wherein the protective film has a side with a flat surface that bears against the laser applicator in the applied state.

8. The protective film as claimed in claim 1, wherein the protective film has a clamping rim surrounding the profile region for fastening the protective film on a film carrier.

9. The protective film as claimed in claim 1, wherein the treatment area is of circular configuration and has a diameter in the range of 8 mm to 11 mm.

10. The protective film as claimed in claim 1, wherein the treatment area is arranged centered on the protective film and wherein the profile region is arranged concentrically with the treatment area.
